(19) United States
(12) Patent Application Publication
(10) Pub. No.: US 2009/0136648 A1
(43) Pub. Date: May 28, 2009

(54) PLATELET AGGREGATION INHIBITOR AND HEALTH FOOD EFFECTIVE IN INHIBITING PLATELET AGGREGATION

(76) Inventors: Hiroyoshi Moriyama, Tokyo (JP); Katsuya Satoh, Oita (JP); Shinsaku Takaoka, Kyoto (JP)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/066,792

(22) PCT Filed: Oct. 17, 2005
(86) PCT No.: PCT/JP2005/019005

§ 371 Date: Mar. 13, 2008

(51) Int. Cl. A61K 38/00; A61P 7/02; A23L 1/30

(52) U.S. Cl. 426/648; 514/2

(57) ABSTRACT

It is intended to provide a platelet aggregation inhibitor which can be easily processed into a pharmaceutical preparation and can be stored for a long time, and a health food effective in inhibiting platelet aggregation. The platelet aggregation inhibitor comprises, as an active ingredient, a degradation product obtained by reacting fibrin with nattokinase, wherein the degradation product has a molecular weight of 10,000 daltons or less. The fibrin is obtained by reacting fibrinogen with thrombin.

### TECHNICAL FIELD

The present invention relates to a platelet aggregation inhibitor, more specifically a platelet aggregation inhibitor comprising, as an active ingredient, a fibrin degradation product obtained by reacting fibrin with nattokinase, and a health food thereof.

### BACKGROUND ART

It has been conventionally known that nattokinase is a thrombolytic enzyme which directly degrades fibrin (blood clot) like plasmin, which is derived from a living body (for example, see Non-Patent Document 1).

In addition, there is described that nattokinase has a platelet aggregation inhibitory effect in Japanese Laid-Open Patent Publication No. 2003-111081 invented and filed by Moriyama and Takaoka among the present inventors.

Non-Patent Document 1: Shinsaku Takaoka: Japan Food Science Vol. 39, September 2000, separate print

### DISCLOSURE OF THE INVENTION

Although it has been known that nattokinase degrades fibrin as above, what kind of action and effect the degradation product has has not been known.

Meanwhile, the present inventors presumed that the degradation product obtained by decomposing fibrin with nattokinase also has a platelet aggregation inhibitory effect since nattokinase has a platelet aggregation inhibitory effect as above.

The present inventors conducted prior art search with regard to this, but there were no prior art documents mentioning whether the degradation product obtained by decomposing fibrin with nattokinase has a platelet aggregation inhibitory effect.

Because the degradation product consists of peptides and is more stable than nattokinase which is an enzyme, it can be easily processed into a pharmaceutical preparation and stored for a long term.

Therefore, the present inventors have conducted intensive studies, supposing that, if the degradation product has a platelet aggregation inhibitory effect, a platelet aggregation inhibitor as well as health food (wholesome food) can be obtained since nattokinase has been eaten as natto or fermented soybeans, for hundreds of years and the safety thereof has been proved, and consequently completed the present invention.

Accordingly, an object of the present invention is to provide a platelet aggregation inhibitor which can be easily processed into a pharmaceutical preparation and stored for a long term, and a health food effective in inhibiting platelet aggregation.

The platelet aggregation inhibitor of the present invention to achieve the above object comprises, as an active ingredient, a degradation product obtained by reacting fibrin with nattokinase.

The platelet aggregation inhibitor of the present invention comprises, as an active ingredient, a degradation product obtained by reacting fibrin with nattokinase, the degradation product having a molecular weight of 10,000 daltons or less.

Furthermore, the health food effective in inhibiting platelet aggregation of the present invention comprises, as an active ingredient, a degradation product obtained by reacting fibrin with nattokinase.

The health food effective in inhibiting platelet aggregation of the present invention comprises, as an active ingredient, a degradation product obtained by reacting fibrin with nattokinase, the degradation product having a molecular weight of 10,000 daltons or less.

The above fibrin is preferably obtained by reacting fibrinogen and thrombin.

In the present invention, the molecular weight of 10,000 daltons or less is specified because it is preferable for processing into a pharmaceutical preparation. That is, if the molecular weight is too large, the product deteriorates in stability and becomes unsuitable for a pharmaceutical preparation.

The present invention has the above-constitution and thereby enables to provide a platelet aggregation inhibitor which is easy to process into a pharmaceutical preparation and can be stored for a long term, and a health food effective in inhibiting platelet aggregation.

### EXAMPLES

Hereinbelow, production process, processing example, and working examples are described.

### (Production Process)

14 ml of a physiologic saline solution (0.85% NaCl) and 4 ml of 9.6 mg/ml fibrinogen (fibrinogen fraction I TypeI-S produced by Sigma, derived from bovine plasma, PRODUCT NUMBER F-8630) were measure into a 100 ml volume conical flask, and after warmed in a constant-temperature water tank at 37±0.3° C. for 10 minutes, added with 1 ml of 20 U/ml thrombin and stirred well. After the solution was allowed to stand at 37° C. for 20 minutes, the solution was added with 1 ml of nattokinase, stirred well and allowed to stand at 37±0.3° C. The solution was stirred well 20, 40, 60, 80 and 100 minutes later after the nattokinase solution was added, and 120 minutes later the solution was boiled in boiling water for 10 minutes to prepare a fibrin degradation product solution (abbreviated as FDP solution). The FDP solution was subjected to ultrafiltration with ULTRA FILTER UNIT USY-5 (molecular weight cutoff: 50,000) manufactured by Advantec, Inc. The obtained permeate was further subjected to ultrafiltration with ULTRA FILTER UNIT USY-1 (molecular weight cutoff: 10,000) to obtain a FDP solution of a molecular weight cutoff: of 10,000 or less. All the reagents were prepared with 0.85% physiologic saline solution.

### (Processing Example)

Dry powders of the above fibrin degradation product can be processed into forms such as capsules, tablets, drinks, granules and pastes and the processing examples are shown below.

As for a soft capsule, for example, 210 mg in total of content fluid obtained by mixing and emulsifying 36.7 mg of fibrin degradation product dry powder, 10 mg of soy bean lecithin, 133.3 mg of soybean oil, 15 mg of beeswax and 15 mg of a fatty acid ester of glycerin is filled into a film consisting of 100 mg of gelatin and 30 mg of glycerin, 130 mg in total, and 340 mg in total weight of a soft capsule can be prepared.

In the same way, as for a hard capsule, 260 mg in total of content fluid obtained by mixing 36.7 mg of fibrin degradation product dry powder, 209.8 mg of dextrin and 13.5 mg of a fatty acid ester of glycerin is filled into a #2 gelatin hardware capsule (70 mg), and 330 mg in total weight of a hard capsules can be prepared.

As for an enteric capsule (containing acid-resistant coating), for example, 210 mg in total of content fluid obtained by mixing and emulsifying 36.7 mg of fibrin degradation product dry powder, 10 mg of soy bean lecithin, 133.3 mg of soybean oil, 15 mg of beeswax and 15 mg of a fatty acid ester of glycerin is filled into a film consisting of 100 mg of gelatin and 30 mg of glycerin, 130 mg in total, and 340 mg in total weight of a soft capsule can be prepared. This is coated with 30 mg of zein coating and 370 mg in total weight of an enteric capsule is prepared.

Besides, the product can be applied for tablets, drinks, granules and pastes, etc.

### Example 1

### Method of Experiment:

Freeze-dried fibrin degradation product solution was diluted to 100 mg, 50 mg, 10 mg solutions with a physiologic saline solution.

Blood was sampled from one man and two women, healthy individuals, and the ability for inhibiting platelet aggregation was measured. Blood was collected from the median vein of forearm with 21G needle using a 3.8% sodium citrate added tube. The obtained blood sample was centrifuged at 180×g for 10 minutes and the supernatant was taken as platelet rich plasma (PRP) and the remaining sample was centrifuged at 1600×g for 15 minutes to obtain platelet poor plasma (PPP). PRP was diluted with PPP and a sample was prepared by adjusting platelet count to 25±3×10⁴/μl. Collagen (MC Medical, Inc.) was used as an aggregation inducer and the final concentration was adjusted to 2 μg/ml. 24 μl of a fibrin degradation liquid or 24 μl of a physiologic saline solution (control) was added to 300 μl of the sample, stirred at 37° C. for one minute and after incubation, 36 μl of the aggregation inducer was added thereto.

A platelet aggregation ability measuring apparatus of particle measuring type using scattered laser light (PA-20 manufactured by Kowa Co., Ltd.) was used for platelet aggregation measurement. PA-20 is an apparatus developed by applying a principle that the intensity of scattered light generated upon hitting fine particles with a straightly traveling light increases in proportion to the square of the particle diameter, and the apparatus enables to calculate the size and number of blood platelet clumps as well as platelet aggregation ratio. The absorbance fell only after clumps consisting of several thousand blood platelets were formed in the conventional absorbance method but this apparatus enables to measure even small clumps consisting of tens of platelets and accordingly it is excellent in detection sensitivity. The blood platelet aggregate size was classified into three classes: 25 mV <(particle size: 9 to 25 μm) <200 mV, 200 mV <medium aggregate (particle size: 25 to 50 μm) <600 mV, 600 mV <large aggregate (particle size: 50 to 70 μm) <2047 mV according to the intensity of scattered laser light [Hoshi K., Zhou X., Terazono M., Satou Y., Yamazaki M., Miyake F., Jpn. J. Clin. Pharmacol. Ther., 32, 223-230 (2001)].

The blood platelet inhibition ratio was calculated by the following expression.

Blood platelet inhibition ratio (%)=(1−X/Y)×100

X: Intensity of scattered laser light or OD (absorbance) after collagen was added and after the fibrin degradation product was added.

Y: Intensity of scattered laser light or OD after collagen was added and before the fibrin degradation product was added.

[Moriyama H., Iizuka T., Nagai M., Hoshi K., Biol. Pharm. Bull., 26, 1361-1364 (2003)].

### Results of Experiment

The following Table 1 shows the inhibition ratio using transmission (OD) and shows that fibrin degradation product at a high concentration (6.70 mg/ml) conspicuously inhibited blood platelet aggregation caused by collagen. In addition, it has been found that the inhibition ratio depends on the concentration of fibrin degradation product.

TABLE 1
Platelet aggregation inhibitory effect by fibrin degradation product

| | Inhibition ratio (%) | | |
|---|---|---|---|
| Inhibition ratio | 6.70 mg/ml | 3.35 mg/ml | 0.67 mg/ml |
| Collagen | 93.5 ± 0.7 | 23.0 ± 7.9 | −0.2 ± 5.2 |
| (2.0 μg/ml) | | | |
| ADP | 64.9 ± 4.1 | 43.8 ± 1.6 | 10.9 ± 9.9 |
| (5.0 μM) | | | |

Average ± Standard error (n = 3)
All concentrations represent the final concentration.

Furthermore, the following Table 2 shows the size distribution of blood platelet aggregates, and the incidence of large clumps was low while the incidence of small clumps was high in the sample to which a fibrin degradation product at a high concentration was added, and it became clear that the formation of the clumps can be controlled with a fibrin degradation product. As a result, it became clear that to fibrin degradation product has a blood platelet inhibitory effect.

TABLE 2
Size distribution of blood platelet
aggregates by the inhibitory effect

| Blood | | | | |
|---|---|---|---|---|
| platelet | Collagen | Fibrin degradation products | | |
| aggregate | 2.0 μg/ml | 6.70 mg/ml | 3.35 mg/ml | 0.67 mg/ml |
| Large | 52.0 ± 10.0 | 9.3 ± 3.5* | 47.3 ± 6.1 | 55.0 ± 2.5 |
| (50-70 μm) | | | | |
| Medium | 18.0 ± 1.5 | 5.7 ± 2.7 | 18.0 ± 2.1 | 18.7 ± 0.9 |
| (25-50 μm) | | | | |
| Small | 30.0 ± 8.5 | 85.0 ± 6.1* | 34.3 ± 4.3 | 26.0 ± 1.7 |
| (9-25 μm) | | | | |

Average ± standard error (n = 3)
*p < 0.05 (Significance test for collagen by Bonferroni multiple comparison)
All concentrations represent the final concentration.

### Example 2

### Method of Experiment:

Vacuum-dried powder of fibrin degradation product solution was diluted to 100 mg, 50 mg and 10 mg solutions with a physiologic saline solution.

Blood was sampled from one man and two women, healthy individuals, and the ability for inhibiting platelet aggregation was measured. Blood was collected from the median vein of forearm with 21G needle using a 3.8% sodium citrate added tube. The obtained blood sample was centrifuged at 180×g for 10 minutes and the supernatant was taken as platelet rich plasma (PRP) and the remaining sample was centrifuged at 1600×g for 15 minutes to obtain platelet poor plasma (PPP). PRP was diluted with PPP and a sample was prepared by adjusting platelet count to 25±3×10⁴/μl. ADP (MC Medical, Inc.) was used as an aggregation inducer and the final concentration was adjusted to 5 μM. 24 μl of a fibrin degradation liquid or 24 μl of a physiologic saline solution (control) was added to 300 μl of the sample, stirred at 37° C. for one minute and after incubation, 36 μl of the aggregation inducer was added thereto.

A platelet aggregation ability measuring apparatus of particle measuring type using scattered laser light (PA-20 manufactured by Kowa Co., Ltd.) was used for platelet aggregation measurement. PA-20 is an apparatus developed by applying a principle that the intensity of scattered light generated upon hitting fine particles with a straightly traveling light increases in proportion to the square of the particle diameter, and the apparatus enables to calculate the size and number of blood platelet clumps as well as platelet aggregation ratio. The absorbance fell only after clumps consisting of several thousand blood platelets were formed in the conventional absorbance method but this apparatus enables to measure even small clumps consisting of tens of platelets and accordingly it is excellent in detection sensitivity. The blood platelet aggregate size was classified into three classes: 25 mV <(particle size: 9 to 25 μm) <200 mV, 200 mV <medium aggregate (particle size: 25 to 60 μm) <600 mV, 600 mV <large aggregate (particle size: 50 to 70 μm) <2047 mV according to the intensity of scattered laser light [Hoshi K., Zhou X., Terazono M., Satou Y., Yamazaki M., Miyake F., Jpn. J. Clin. Pharmacol. Ther., 32, 223-230 (2001)].

The blood platelet inhibition ratio was calculated by the following expression.

Blood platelet inhibition ratio (%)=(1−X/Y)×100

X: Intensity of scattered laser light or OD (absorbance) after ADP was added and after the fibrin degradation product was added.

Y: Intensity of scattered laser light or OD after ADP was added and before the fibrin degradation product was added.

[Moriyama H., Iizuka T., Nagai M., Hoshi K., Biol. Pharm. Bull., 26, 1361-1364 (2003)].

### Results of Experiment

The above Table 1 shows the inhibition ratio using transmission (OD) and shows that fibrin degradation product at a high concentration (6.70 mg/ml) conspicuously inhibited blood platelet aggregation caused by ADP. In addition, it has been found that the inhibition ratio depends on the concentration of fibrin degradation product.

Furthermore, the following Table 3 shows the size distribution of blood platelet aggregates, and the incidence of large clumps of blood platelets was low while the incidence of small clumps was high in the sample to which a fibrin degradation product at a high concentration was added, and it became clear that the formation of the clumps can be controlled with a fibrin degradation product. As a result, it became clear that a fibrin degradation product has a blood platelet inhibitory effect.

TABLE 3
Size distribution of blood platelet
aggregates by the inhibitory effect

| Blood | | | | |
|---|---|---|---|---|
| platelet | ADP | Fibrin degradation products | | |
| aggregate | 5.0 μM | 6.70 mg/ml | 3.35 mg/ml | 0.67 m/ml |
| Large | 56.7 ± 2.6 | 7.0 ± 2.3* | 29.0 ± 10.4 | 49.7 ± 8.8 |
| (50-70 μm) | | | | |
| Medium | 19.7 ± 0.9 | 16.3 ± 1.2 | 20.3 ± .2.6 | 23.7 ± 4.4 |
| (26-50 μm) | | | | |
| Small | 24.0 ± 2.5 | 76.7 ± 3.5* | 50.7 ± 11.3 | 30.7 ± 5.7 |
| (9-25 μm) | | | | |

Average ± standard error (n = 3)
*p < 0.05 (Significance test for ADP by Bonferroni multiple comparison)
All concentrations represent the final concentration.

1. A platelet aggregation inhibitor characterized by comprising, as an active ingredient, a degradation product obtained by reacting fibrin with nattokinase.

2. A platelet aggregation inhibitor characterized by comprising, as an active ingredient, a degradation product obtained by reacting fibrin with nattokinase, wherein the degradation product has a molecular weight of 10,000 daltons or less.

3. Health food effective in inhibiting platelet aggregation characterized by comprising, as an active ingredient, a degradation product obtained by reacting fibrin with nattokinase.

4. A health food effective in inhibiting platelet aggregation characterized by comprising, as an active ingredient, a degradation product obtained by reacting fibrin with nattokinase, wherein the degradation product has a molecular weight of 10,000 daltons or less.

5. The platelet aggregation inhibitor according to claim 1 characterized in that the fibrin is obtained by reacting fibrinogen and thrombin.

6. The health food effective in inhibiting platelet aggregation according to claim 3 characterized in that the fibrin is obtained by reacting fibrinogen and thrombin.

7. The platelet aggregation inhibitor according to claim 2 characterized in that the fibrin is obtained by reacting fibrinogen and thrombin.

8. The health food effective in inhibiting platelet aggregation according to claim 4 characterized in that the fibrin is obtained by reacting fibrinogen and thrombin.
